(19) United States
(12) Patent Application Publication
(10) Pub. No.: US 2023/0323461 A1
(43) Pub. Date: Oct. 12, 2023

(54) METHODS AND SYSTEMS FOR STRATIFYING INFLAMMATORY BOWEL DISEASE PATIENTS

(71) Applicants: CEDARS-SINAI MEDICAL CENTER, Los Angeles, CA (US); The Broad Institute, Inc., Cambridge, MA (US); The General Hospital Corporation, Boston, MA (US)

(72) Inventors: Dermot P. MCGOVERN, Los Angeles, CA (US); Dalin LI, Los Angeles, CA (US); Yunfeng RUAN, Cambridge, MA (US); Ruize Liu, Cambridge, MA (US); Mark DALY, Boston, MA (US); Hailiang HUANG, Boston, MA (US)

(21) Appl. No.: 18/190,731

(22) Filed: Mar. 27, 2023

Related U.S. Application Data
(63) Continuation of application No. PCT/US2021/053515, filed on Oct. 5, 2021.
(60) Provisional application No. 63/087,771, filed on Oct. 5, 2020.

(51) Int. Cl. C12Q 1/6883; G16B 20/20; G01N 33/68; G16H 50/20; G16B 40/20

(52) U.S. Cl. CPC C12Q 1/6883; G16B 20/20; G01N 33/6893; G16H 50/20; G16B 40/20; G01N 2800/065; C12Q 2600/156; C12Q 2600/158

(57) ABSTRACT

Provided herein are methods and systems for use in identifying a subject with inflammatory bowel disease based on certain criteria, including genetics or serological markers measured in a sample obtained from the subject. Also provided are methods and systems for treatment of the inflammatory bowel disease in the subject. The systems described herein may include polygenic risk score (PRS), which is useful for determining the relative risk of the subject as compared with a reference population.

### CROSS REFERENCE

This application is a continuation of International Application No. PCT/US2021/053515, filed Oct. 5, 2021, which claims the benefit of U.S. Provisional Patent Application No. 63/087,771 filed Oct. 5, 2021, each of which is herein incorporated by reference in its entirety.

### STATEMENT AS TO FEDERALLY SPONSORED RESEARCH

This invention was made with government support under DK046763 and DK062413 awarded by National Institutes of Health. The government has certain rights in the invention.

### BACKGROUND

The inflammatory bowel diseases (IBD) are a group of human immune-mediated and inflammatory disorders affecting over 1.6 million Americans. IBD is a term for two complex disorders: Crohn's disease (CD) and ulcerative colitis (UC), both have been shown to be highly heritable. CD is a chronic inflammatory disease that can affect any part of the gastrointestinal tract but is mostly localized to the terminal ileum and colon. CD is characterized by discontinuous inflammation that can be transmural whereas UC is restricted to the colon, is continuous and the inflammation is usually limited to the mucosal layer.

Clinically, the heterogeneous presentation of IBD subtypes is a major diagnostic challenge, as distinguishing CD from UC can be difficult in 5-15% of patients. Diagnostic uncertainty and subsequent misclassification in IBD are associated with higher rates of complicated disease, relapse, and cancer. Furthermore, while some therapies are universally effective, others show clinical efficacy in either CD (e.g. methotrexate) or UC (e.g. mesalamine, tofacitinib) exclusively. Modern molecular techniques have paved the way for reclassification of diseases, whereby individuals with similar molecular etiopathogenesis are likewise grouped irrespective of their ‘classical’ diagnosis. Applying this concept to IBD might ultimately improve prediction of clinical outcomes and response to treatment.

### SUMMARY

Provided herein are methods and systems for diagnosing, prognosing and/or treating inflammatory bowel diseases, such as Crohn's disease and ulcerative colitis.

Disclosed herein, in certain aspects is a method of diagnosing a subject with an inflammatory bowel disease, the method comprising: (a) assaying a biological sample of the subject to generate a genetic dataset comprising genetic data; (b) processing the genetic dataset at a genetic locus to determine quantitative measure of the genetic locus, wherein the genetic locus comprises a gene associated with Crohn's disease, thereby producing a Crohn's disease profile of the biological sample of the subject; and (c) applying a prediction model to the Crohn's disease profile to diagnose the inflammatory bowel disease in the subject, or a likelihood that the subject will develop the inflammatory bowel disease. In some embodiments, the genetic locus is at a NOD2 gene. In some embodiments, the method further comprises: (d) assaying the biological sample of the subject to generate a serological dataset comprising serology data; (e) processing the serological data set comprising a presence or a level of a serological marker, thereby refining the Crohn's disease profile of the biological sample of the subject; and (f) applying the prediction model to the Crohn's disease profile refined in (e) to diagnose the inflammatory bowel disease in the subject, or a likelihood that the subject will develop the inflammatory bowel disease. In some embodiments, the serological marker comprises anti-Saccharomyces Cerevisiae antibodies (ASCA IgG and IgA), perinuclear anti-nuclear cytoplasmic antibody (pANCA), anti-flagellin (anti-CBir1), anti-outer membrane porin C (anti-OmpC) or anti-Pseudomonas fluorescens-associated sequence I2 (anti-I2), or a combination thereof. In some embodiments, applying the prediction model is performed to further diagnose the inflammatory bowel disease location comprising an ileum, a colon, or ileocolonic region of an intestine of the subject. In some embodiments, the method further comprises administering to the subject a Crohn's disease therapy, provided the inflammatory bowel disease diagnosed in the subject is Crohn's disease.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows variance explained by genome-wide or locus-based genetic prediction models. P-value cut-off of 0.1 was used to build the models except for NOD2 for which the putative causal variants from fine-mapping were used (*). Models were tested on non-Jewish (NJ) and Jewish (J) CEDAR samples. All: the genome-wide model; Other: models using genomic regions other than regions listed in the figure. Error bar: 95% confidence interval.

FIGS. 2A-4B shows the marginal and conditional variance explained by genetics, serology, smoking, and their joint models. FIG. 2A depicts the marginal variance explained by genetics, serology serum, smoking, and their combinations. Error bar indicates 95% confidence interval. FIG. 2B depicts the marginal and conditional variance explained by genetics, serum, smoking and the relative contribution from each factor within them.

FIG. 3 shows the accuracy of the genetic model test on the colonic CD vs UC and on small bowel CD vs UC. Error bar: 95% confidence interval.

FIGS. 4A-4B shows a genetic association analysis for CD versus UC using IIBDGC non-Jewish samples (with CEDAR samples excluded). FIG. 4A is a Manhattan plot with top associations annotated with a gene within ±300 kb. FIG. 4B is a regional association plot for the MEC locus post imputation.

FIG. 5 depicts the variance explained by genome-wide or locus-based genetic prediction models. P-value cut-off of 0.1, 0.01 and 0.001 were used respectively to build the models except for NOD2 for which the putative causal variants from fine-mapping were used (*). The models were tested on non-Jewish (NJ) and Jewish (J) CEDAR samples. All: the genome-wide model; Other: model using genomic regions other than regions listed in the figure. Error bar: 95% confidence interval.

FIG. 6 depicts the variance explained by the NOD2 and MEC loci using different genetic models. The genetic models were tested on non-Jewish (NJ) and Jewish (J) CEDAR samples respectively. NOD2_PT: genetic model using the P+T variants (P-value cut-off at 0.1) for NOD2; NOD2_FM: genetic model using the fine-mapped causal variants for NOD2, MEC TOP: genetic model using the most significant variant from the MEC locus; MEC_PT: genetic model using the P+T variants (P-value cut-off at 0.1) for the MEC locus; MEC_FM: genetic model using the fine-mapped causal variants for the MEC locus. Error bar: 95% confidence interval.

FIG. 7 depicts the performance of models tested on IIBDGC and CEDAR samples. The genetic model trained using 50% IIBDGC samples were tested on the remaining 50% IIBDGC samples (IIBDGC) and the CEDAR samples (CEDAR). NJ: non-Jewish; J: Jewish; Error bar: 95% confidence interval.

FIG. 8 depicts the variance explained by models with serum, smoking and combined models on location of colonic CD vs UC and small bowel CD vs UC. Error bar: 95% confidence interval.

FIG. 9 shows a computer system that is programmed or otherwise configured to implement methods provided herein.

### DETAILED DESCRIPTION

The inflammatory bowel diseases (IBD) are a group of polygenic immune-mediated disorders of the gastrointestinal tract. Ulcerative colitis (UC) and Crohn's disease (CD) are two etiologically related but distinct IBD disorders. Due to the heterogeneous presentation of IBD, discriminating CD from UC can be challenging for some patients using the conventional clinical approaches. A novel molecular based prediction model was designed and evaluated aggregating genetics, serology and tobacco smoking information to assist the diagnosis of CD and UC for IBD patients. The genetics component was trained on a large-scale cohort (CD: 15,987, UC: 12,613) and accounted for 19.3% of variance explained, among of which NOD2, the MHC locus, PTGER4, ATG16L1, IL23R, PHTF1, IRF1, HNF4A and RNF186 made the greatest contributions. The serology component (ASCA (IgA/IgG), ANCA, anti-Cbir1, anti-OmpC, and anti-I2) and smoking information (never, ex-smoker and current) accounted for 38.8% and 1.1% of variance explained respectively. Despite moderate overlap among them, genetics, serology and smoking each makes independent contributions to distinguish IBD subtypes, and together they explained 45.6% of variance. Molecular-based models combining genetics, serology and smoking information could complement current diagnostic strategies and help classify patients based on biologic state rather than imperfect clinical parameters.

Serology markers, including anti-Saccharomyces cerevisiae antibodies (ASCA, largely CD-specific) and perinuclear, DNAseI sensitive, “atypical” neutrophil cytoplasmic antibodies (p-ANCA, largely UC-specific) are specific but non-sensitive IBD biomarkers. When used in combination, they are highly specific for UC and CD. However, as almost half of patients are negative for both markers, they are uninformative in many cases. Smoking status is the only reliably associated environmental factor that has differential effects in CD and UC, as current smokers are at higher risk for CD (OR=1.76) and are protected against UC (OR=0.58).

Genome-wide association studies (GWAS) have been successful in finding genetic loci associated with IBD, with more than 200 associations with IBD established in the past years including NOD2, IL23R, and the major histocompatibility complex (MHC) locus. Many of these genetic associations preferentially implicate one IBD subtype to the other. For example, the most robust IBD genotype-phenotype relationship in IBD, a NOD2 frameshift variant (r55743293), significantly increases one's risk to CD but has almost no influence on one's risk to UC. Despite its significance and strong effect, this variant, when used alone, has limited applications in classifying IBD patients into clinically relevant subgroups because of its low population prevalence (4% in Europeans). However, when genetic information is aggregated across the genome, in the form of polygenic risk scores (PRS), prediction accuracy can increase drastically.

Previous studies have shown that genetics, serology, and tobacco smoking each make their own contribution to inform about IBD subtypes, but individually, they have limited sensitivity and/or specificity. An earlier study also showed that jointly modeling all these factors may have improved accuracy, but was limited in terms of the sample size, the lack of genome-wide genetics data, and the failure to control for population structure. A joint prediction model was developed using an IBD cohort that is 30-40 times larger than the previous study, fully and robustly exploring the individual and the joint contributions from these factors in diagnosing CD and UC for IBD patients.

Predicting CD and UC Using the Genetics Information

The ability of using genetic information to predict whether an individual's diagnosis is CD or UC was assessed. To do this, the genetics prediction model was trained on the non-Jewish IIBDGC cohort (with CEDAR samples excluded) and evaluated its performance on CEDAR samples (Method). 1,000 bootstraps (sample with replacement) were performed on CEDAR samples to evaluate the variance of the estimate. Non-Jewish and Jewish CEDAR samples were evaluated independently.

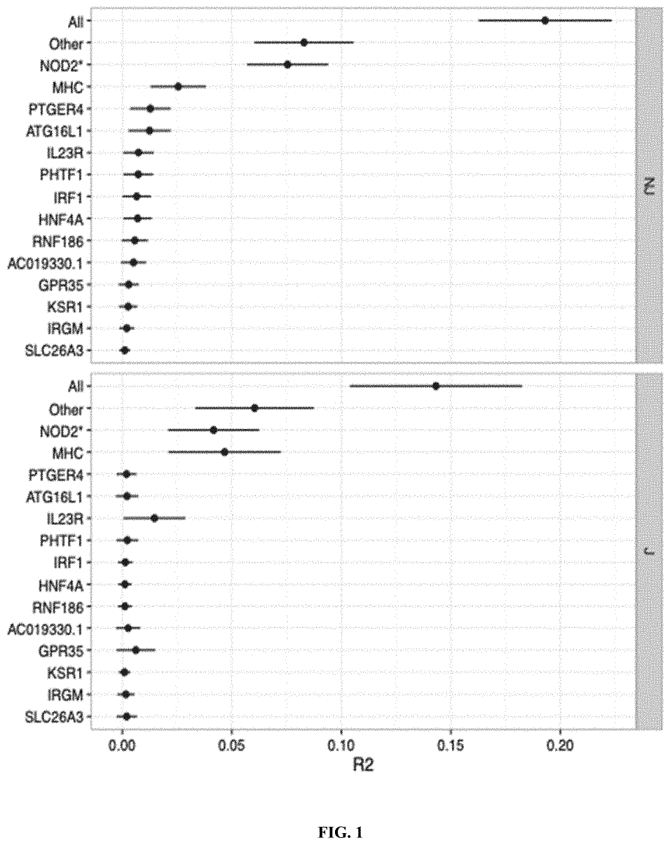

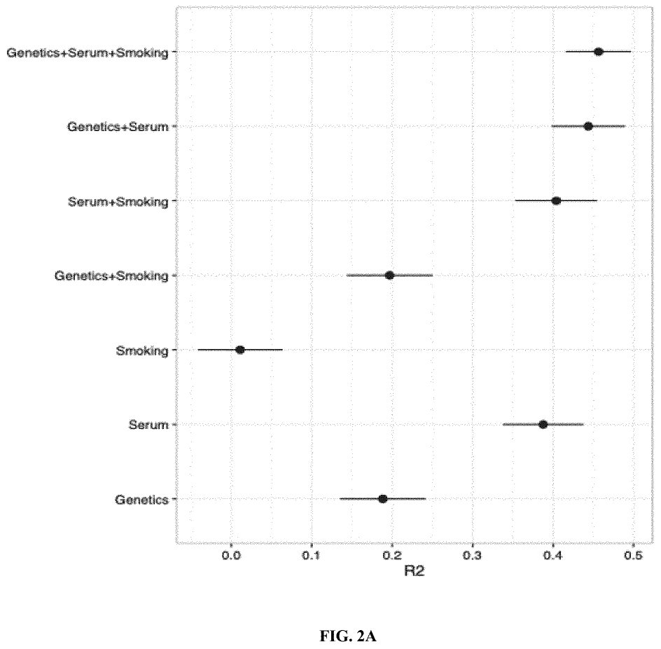

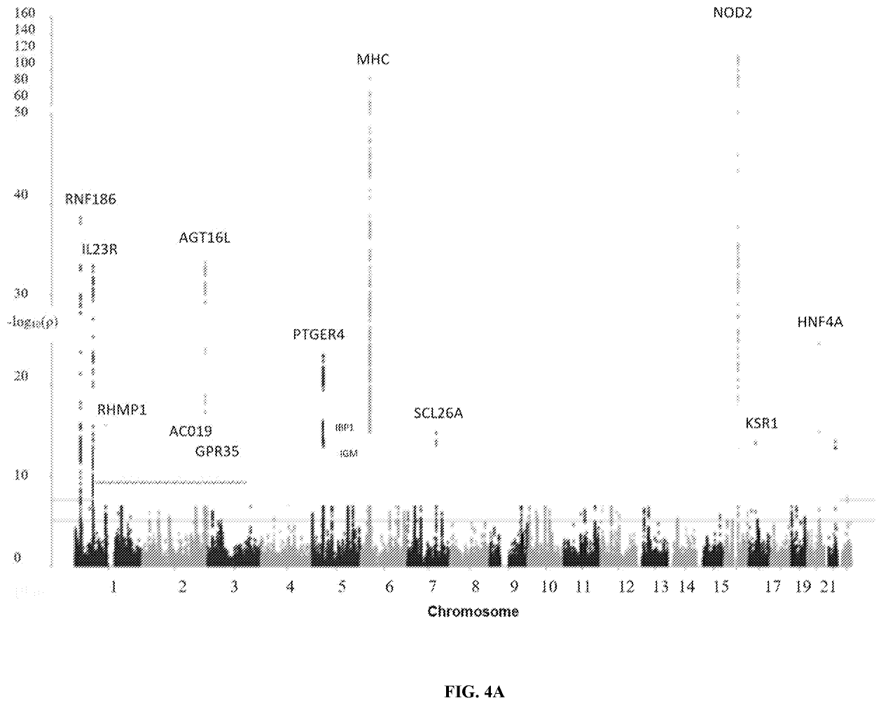

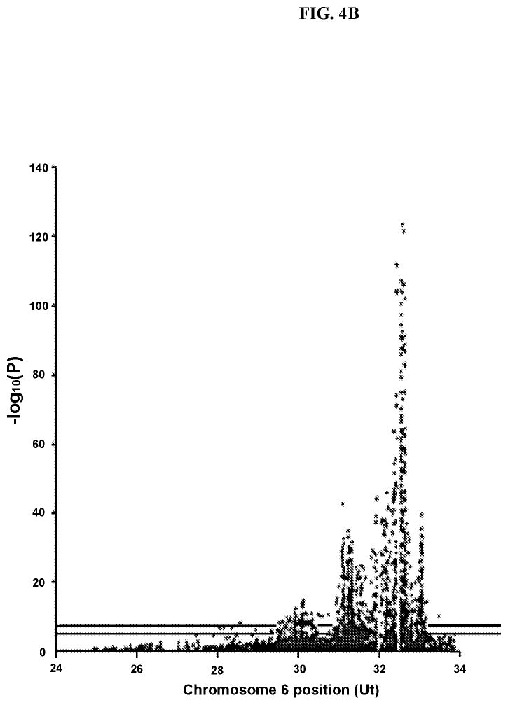

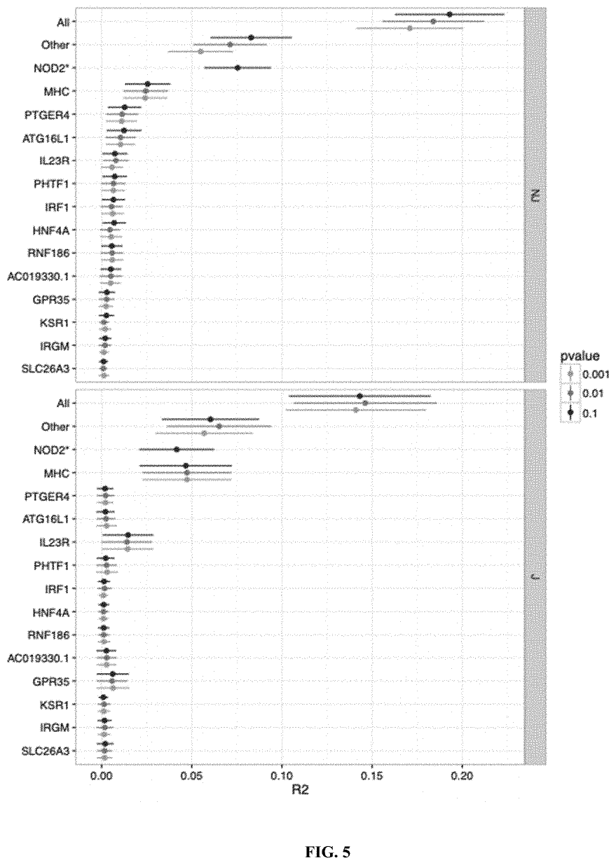

To build the genetic prediction model, performed genome-wide association analysis (GWAS) was performed with CD and UC as the trait (FIG. 4A), followed by clumping and thresholding (P+T) using a cut-off of P-value<0.1 (Association analysis, Method). Additional P-value cut-offs were evaluated and were found to make no significant difference (FIG. 5). The PRS for each sample was calculated from the P+T results (Polygenic risk score, Method), with the exception of NOD2 for which its putative causal variants from fine-mapping were used to fully capture its genetic contribution (discussed later). The variance explained for non-Jewish samples was 0.193±0.030 (FIG. 1, error bar indicates 95% confidence interval. Same for all unless specified otherwise). For Jewish samples the variance explained slightly dropped to 0.143±0.039.

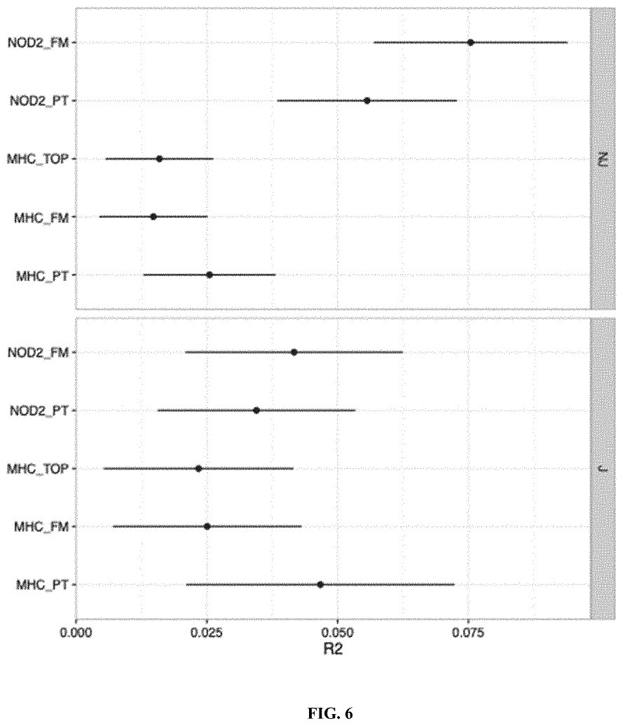

Genes making the greatest contribution to the genetic model include NOD2, the MHC locus, PTGER4, ATG16L1, IL23R, PHTF1, IRF1, HNF4A and RNF186, which are the top significant genetic loci from GWAS (FIG. 4A). But in aggregation the 13 genetic loci mapped from the top 30 most significant associations explain <50% of the total variance explained, consistent with the polygenic genetic architecture of IBD (FIG. 1) and justifies the use of genome-wide data in the genetic model. For the top genetic loci, the prediction accuracy can be improved if the genetic risk model uses the putative causal variants from fine-mapping rather than variants from P+T, as the former has a better signal-to-noise ratio. By using results from a published fine-mapping study (Huang, H. et al. Fine-mapping inflammatory bowel disease loci to single-variant resolution. Nature 547, 173-178 (2017)), the variance explained in the NOD2 locus improved from 0.055±0.017 to 0.075±0.018 (FIG. 6). For other genetic loci including the MHC locus, the variance explained stays roughly the same (FIG. 6). Therefore, the fine-mapped variants from NOD2 and the P+T variants from other loci were used for the final genetic model.

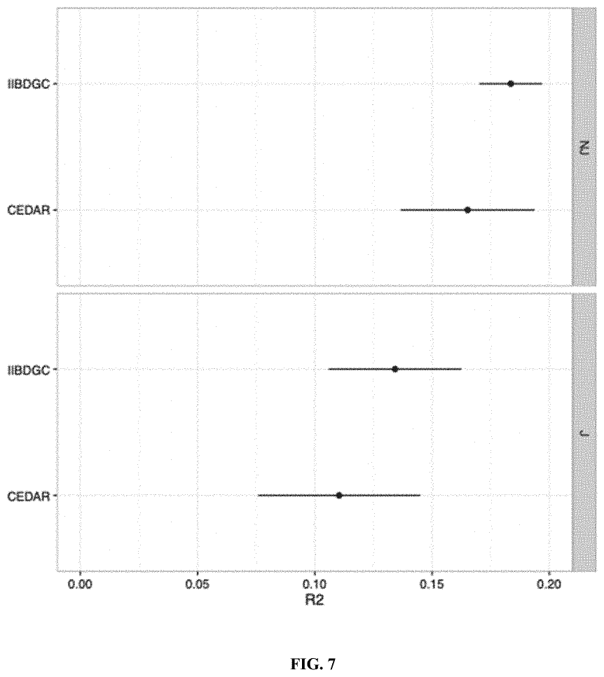

All analyses described above used CEDAR samples as testing and IIBDGC samples as training. As a validation, bootstrap was used to evaluate our genetics model on the IIBDGC samples. To ensure a fair comparison, the genetics model was trained using 50% IIBDGC samples and the model was tested on the remaining 50% IIBDGC samples and the CEDAR samples respectively. This analysis was repeated 1,000 times. The variance explained has no significant difference, confirming the robustness of our results (FIG. 7).

Contribution from Serology and Smoking

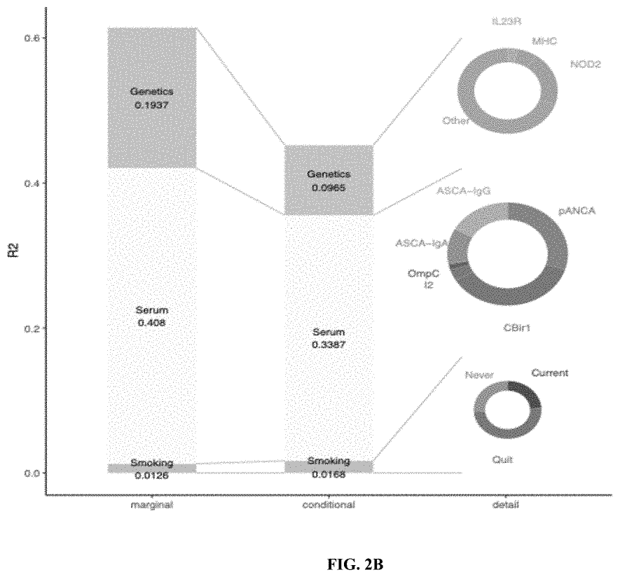

The contribution from serology biomarkers (ASCA, pANCA, anti-CBir1, OmpC, and 12) and smoking status to discriminate CD from UC was explored. The analysis was only performed on the CEDAR samples as serology and smoking data is not available in the IIBDGC samples. 1,000 cross-validations were performed, with 50% samples used as training and the remaining samples as testing (Method). The serology biomarkers explain 0.388±0.050 of the total variance, and smoking status explains 0.011±0.052 of the total variance (FIG. 2A). The model combining all the data (genetics, serology, and smoking status) has the best prediction accuracy, explaining 0.456±0.041 of total variance (FIG. 2A).

How each factor contributes to the prediction accuracy conditional on other factors was then analyzed. The variance explained by genetics reduced from 0.19 to 0.09, and serology from 0.41 to 0.34 (FIG. 2B). The conditional contribution from genetics is, however, still significant. The contribution from smoking status stayed similar (variance explained from 0.012 to 0.016). (FIG. 2B).

Almost 21% of the variance explained by the genetics model was accounted for by NOD2, and 6% was accounted for by the MHC locus (FIG. 2B). The remaining genome region accounted for −70%, consistent with the polygenic nature of IBD. Among serology markers, CBir1 makes the largest contribution (36%) and the majority (95%) of the variance explained was accounted for by the top 4 serological markers: CBir1, ANCA, ASCA-IgG, and ASCA-IgA.

Using Genetic, Serology and Smoking Status to Predict the Disease Location

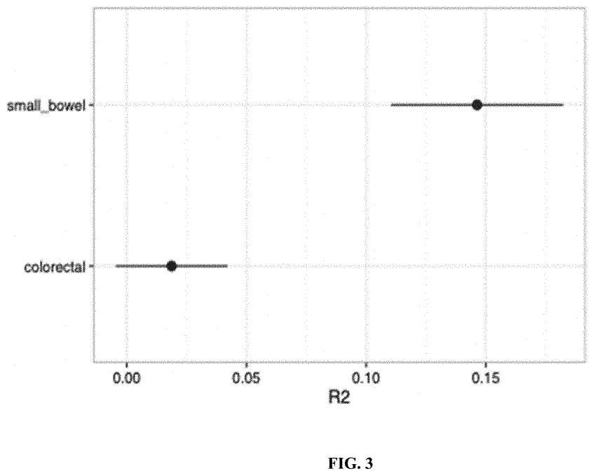

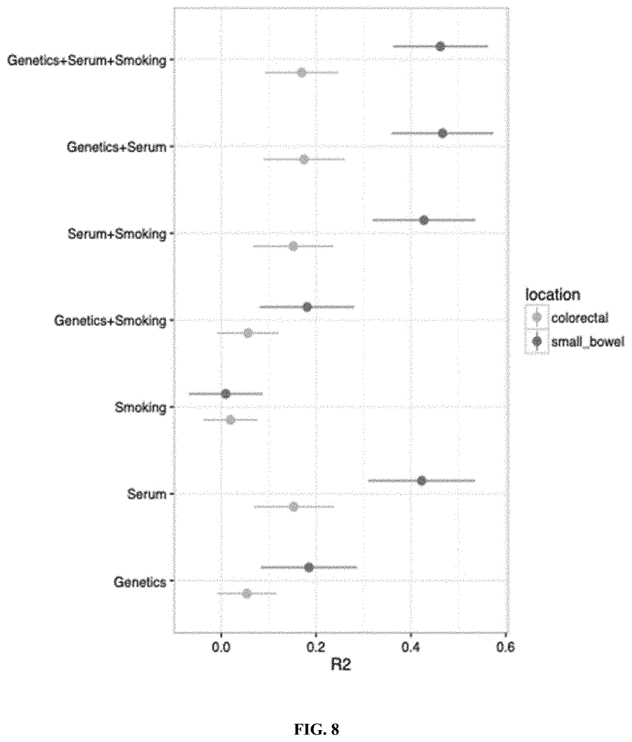

Depending on the disease location, CD patients can largely be classified into colonic CD and small bowel CD. The ability of the genetic model to predict the two CD subtypes against UC was evaluated. To make a fair comparison, the IIBDGC non-Jewish samples was sampled such that the training data has the same number of colonic CD and small bowel CD: 2,271 colonic CD, 2,271 small bowel CD and 2,300 UC. The testing data was sampled from CEDAR with 276 colonic CD and 276 UC for predicting colonic CD v.s. UC, and 1,100 small bowel CD and 1,100 UC for predicting small bowel CD v.s. UC. The analysis was repeated 1,000 times to evaluate the variance. The genetic model explained 0.146±0.036 of total variance for small bowel CD vs UC (FIG. 3) but only 0.019±0.023 for colonic CD versus UC. The same drop in prediction accuracy was observed when serology was used in the prediction model (FIG. 8).

The Clinical Application with the Combined Model

Prediction models were investigated using genetic, serology markers and smoking status to facilitate the diagnosis of CD and UC patients. Samples from IIBDGC and CEDAR were used to train and validate those models. A model combining the molecular and environmental information could complement current diagnostic strategies and help classify patients based on biologic state rather than imperfect clinical parameters.

The serology information makes the greatest contribution. Anti-CBir1 was uniquely associated with CD and ANCA was predominantly associated with UC. It was demonstrated that although the differential UC/CD effect of smoking is well-established in our study, it only explains a small percent of variance. Genetic information, even conditioned on serology measures, makes significant contributions to the prediction accuracy.

One strategy to improve the accuracy of the genetic model is to use the fine-mapped causal variants as they better capture the genuine causal genetic effects. The benefit for the NOD2 gene was demonstrated (FIG. 6). In loci with less power, P+T might be a better choice as P+T is less affected by power because it includes variants below the significance thresholds, which have been shown to improve the prediction accuracy. Additionally, the long-range LD structure in MEC could also make fine-mapping more challenging, reducing the gain from using their fine-mapped causal variants.

Colon is a tissue that can be affected by both CD and UC. Previous work has shown that ileal CD, colonic CD and UC can be distinguished by their genetic risk score, with colonic CD showing a genetic risk score ‘lying’ between ileal CD and UC. Our study confirmed that colonic CD is more similar to UC than non-colonic CD pathologically and in their genetic and serology profiles.

### Computer Systems

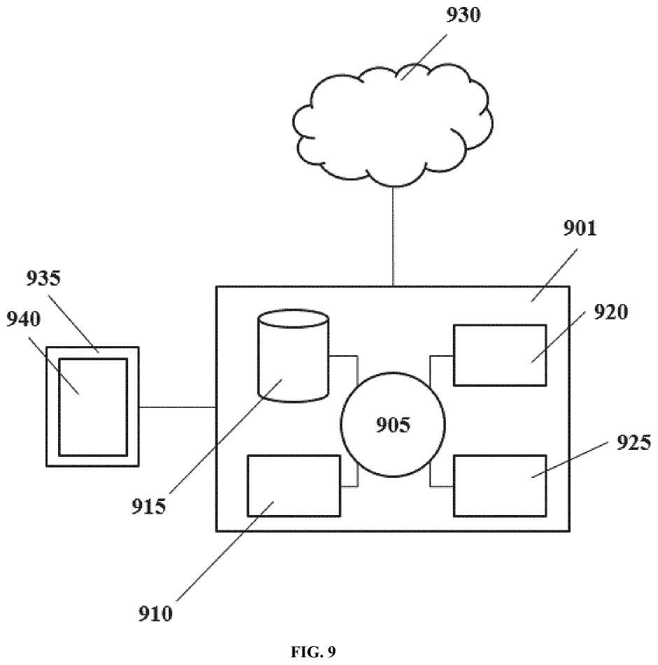

The present disclosure provides computer systems that are programmed to implement methods of the disclosure. FIG. 9 shows a computer system 901 that is programmed or otherwise configured to analyze a biological sample (e.g., to diagnose, prognose or treat an inflammatory bowel disease). The computer system 901 can regulate various aspects of the present disclosure, such as, for example, (a) receiving data (e.g., genetic data, serological data), (b) processing the data to produce a profile (e.g., a Crohn's disease profile) of the biological sample, and (c) applying a prediction model to the Crohn's disease profile to diagnose or prognose a subject with an inflammatory bowel disease or subtype (e.g., Crohn's disease). The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, the diagnosis or prognosis of the subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example: (a) process a dataset (e.g., genetic data set, serological data set, clinical data set) to determine quantitative measure of a marker (e.g., genetic locus, serological marker, clinical parameter) associated with Crohn's disease, thereby producing a Crohn's disease profile of the biological sample of the subject; and (b) applying a prediction model to the Crohn's disease profile to diagnose the inflammatory bowel disease in the subject, or a likelihood that the subject will develop the inflammatory bowel disease.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject” or “individual” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

In some embodiments, the subject is a “patient”, which in some cases refers to a subject that has been diagnosed with a disease or condition described herein (e.g. Inflammatory Bowel Disease).

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Examples

The following examples are included for illustrative purposes only and are not intended to limit the scope of the invention.

### Example 1: Methods and Materials

Sample Characteristics

IIBDGC: individual-level genotypes were obtained from the International Inflammatory Bowel Disease Genetics Consortium (IIBDGC). After excluding samples from CEDAR, there were 17,495 CD and 13,728 UC individuals (Table 1). All samples have gone through quality control as described in Huang, H. et al. Fine-mapping inflammatory bowel disease loci to single-variant resolution. Nature 547, hereinafter incorporated by reference.

TABLE 1
Sample information used in the following studies

| IIBDGC samples | | | Cedars Samples | |
|---|---|---|---|---|
| sample size | Non-Jewish | Jewish | Non-Jewish | Jewish |
| CD | 15,987 | 1,508 | 1947 | 990 |
| -----colonic | 2,271 | 139 | 276 | 118 |
| -----small bowel | 13,716 | 1369 | 1671 | 872 |
| UC | 12,613 | 1,115 | 1100 | 541 |

CEDAR: Participants in CEDAR were recruited at the IBD and pediatric IBD Centers at Cedars-Sinai Medical Center, Los Angeles, USA. After filtering out non-European ancestry and IBDU participants, there are 3,462 CD and 2,044 UC (Table 1, Cedars Samples). Individual-level genotypes, serology, and smoking status are available in CEDAR. QC for CEDAR samples was described in Lew, D. et al. Genetic associations with adverse events from anti-tumor necrosis factor therapy in inflammatory bowel disease patients. World J. Gastroenterol. 23, 7265-7273 (2017), hereinafter incorporated by reference.

All analyses in this study were performed on a set of variants that are shared by both IIBDGC and CEDAR samples post-QC (129,199 variants). Due to the high density design of the immunochip, imputation was not performed except for the MHC locus, for which variants were imputed within the class I and class II HLA genes at the level of classical HLA alleles and amino acids in IIBDGC and CEDAR samples using Beagle21 (version 5) and the T1DGC MHC reference panel22 (8,961 variants and 5,225 samples). 8,312 variants and HLA alleles with imputation quality >0.6 in both samples, plus 936 immunochip variants by design were used for further analysis.

Smoking Status

Tobacco smoking was considered as a categorical variable, with three levels: (a) IBD patients who have never smoked; (b) previous smokers who quit before their IBD diagnosis; and (c) patients who were smokers at the time of IBD diagnosis. These definitions were uniformly used in all of the cohorts.

Serologic Ascertainment

Serum immune responses: anti-Saccharomyces Cerevisiae antibodies (ASCA IgG and IgA), perinuclear anti-nuclear cytoplasmic antibody (pANCA), anti-flagellin (anti-CBir1), anti-outer membrane porin C (anti-OmpC) and anti-Pseudomonas fluorescens-associated sequence I2 (anti-I2) were analyzed by ELISA on the CEDARS cohort as previously described in Lew, D. et al. Genetic associations with adverse events from anti-tumor necrosis factor therapy in inflammatory bowel disease patients. World J. Gastroenterol. 23 7265-7273 (2017) and Jia, X. et al. Imputing amino acid polymorphisms in human leukocyte antigens. PLoS One 8, e64683 (2013), herein incorporated by reference. Serologic markers were typically obtained at study enrollment and consent in the Cedars-Sinai IBD Centers. All assays were performed blindly without knowledge of patient clinical characteristics.

Principal Component Analysis and Ancestry

Principal components (PC) for IIBDGC samples were taken from a previous studies (Huang, H. et al. Fine-mapping inflammatory bowel disease loci to single-variant resolution. Nature 547, 173-178 (2017)). For the CEDAR samples, the following steps were used to generate the principal components: 1) variants were removed within the MHC region, with MAF<0.05, with a call rate <0.99, or in violation of Hardy Weinberg equilibrium with P-value <1 e-5. This strict quality filter allows us to ensure the top PCs capture the population structure rather than genotyping artifacts. 2) variants were LD pruned with pairwise R2=0.1, window size=100 variants and step size=5 variants; this was repeated three times to address the complex LD structure. 3) after pruning, 14,963 variants were used for PC analysis by PLINK.

Jewish ethnicity was estimated based on the Human Genome Diversity Project (HGDP) reference panel and a local Jewish non-IBD reference cohort using Admixture. Individuals with >75% Jewish components were classified as Jewish.

Association Analysis

Genome-wide association analysis was performed on 15,987 CD and 12,613 UC samples from IIBDGC. Genetic Jewish samples and CEDAR samples was excluded in the association analysis, and only used variants that are shared with the post-QC CEDAR samples as discussed in Sample characteristics. Logistic regression was performed on variants having minor allele frequency >0.5% (114,146 variants) with the top ten PCs as covariates using PLINK 1.9. 1,944 variants were associated with CD and UC at genome-wide significance (FIG. 4A, Table 2). In the MHC locus, the imputed HLA variants was included (Sample characteristics) to achieve the maximum coverage (8,312 imputed variants/HLA alleles and 936 variants from immunochip), among which 2,350 variants are significantly associated with subtypes of IBD (FIG. 4B).

TABLE 2
Genes implicated by top significant variants

| CHR | SNP | SNP BP | P | Gene (within +/−300 kb) | Gene CHR | Gene region |
|---|---|---|---|---|---|---|
| 16 | rs5743293 | 50763781 | 8.41E−156 | NOD2 | 16 | 50727507-50766990 |
| 16 | rs5743289 | 50756774 | 3.40E−116 | NOD2 | 16 | 50727507-50766990 |
| 16 | rs2066845 | 50756540 | 1.18E−52 | NOD2 | 16 | 50727507-50766990 |
| 16 | rs7194886 | 50725193 | 3.46E−46 | NOD2 | 16 | 50727507-50766990 |
| 1 | rs6426833 | 20171860 | 3.25E−39 | RNF186 | 1 | 20140522-20141771 |
| 16 | rs2357623 | 50694011 | 2.49E−36 | NOD2 | 16 | 50727507-50766990 |
| 2 | rs6752107 | 234161448 | 2.53E−34 | ATG16L1 | 2 | 234160217-234204320 |
| 1 | rs1004822 | 67668551 | 3.51E−34 | IL23R | 1 | 67604590-67725662 |
| 20 | rs6017342 | 43065028 | 2.45E−25 | HNF4A | 20 | 42984441-43061485 |
| 16 | rs1077861 | 50759547 | 4.05E−24 | NOD2 | 16 | 50727507-50766990 |
| 5 | rs9283753 | 40490609 | 4.63E−24 | PTGER4 | 5 | 40680032-40696962 |
| 16 | rs1990623 | 50565970 | 4.33E−23 | NOD2 | 16 | 50727507-50766990 |
| 5 | rs348594 | 40323938 | 9.80E−23 | PTGER4 | 5 | 40680032-40696962 |
| 2 | rs6758145 | 234165157 | 4.58E−19 | ATG16L1 | 2 | 234160217-234204320 |
| 1 | rs2066130 | 20244408 | 8.28E−19 | RNF186 | 1 | 20140522-20141771 |
| 1 | rs11209026 | 67705958 | 6.40E−18 | IL23R | 1 | 67604590-67725662 |
| 5 | rs2371685 | 40392226 | 9.81E−17 | PTGER4 | 5 | 40680032-40696962 |
| 1 | rs6679677 | 114303808 | 1.64E−16 | PHTF1 | 1 | 114239824-114302165 |
| 1 | rs10889674 | 67717528 | 3.76E−16 | IL23R | 1 | 67604590-67725662 |
| 7 | rs6466198 | 107480126 | 1.91E−15 | SLC26A3 | 7 | 107405912-107443678 |
| 21 | rs9977672 | 40463283 | 1.56E−14 | — | — | — |
| 17 | rs10775412 | 25869033 | 2.38E−14 | KSR1 | 17 | 25799036-25950718 |
| 1 | rs4655215 | 20137714 | 2.70E−14 | RNF186 | 1 | 20140522-20141771 |
| 2 | rs34119476 | 199584179 | 3.32E−14 | AC019330.1 | 2 | 199417919-199637080 |
| 2 | rs4676406 | 241579108 | 4.09E−14 | GPR35 | 2 | 241544825-241570676 |
| 5 | rs12521868 | 131784393 | 1.18E−13 | IRF1 | 5 | 131817301-131826465 |
| 16 | rs6500315 | 50508101 | 1.77E−13 | NOD2 | 16 | 50727507-50766990 |
| 16 | rs1528602 | 50999201 | 5.78E−13 | NOD2 | 16 | 50727507-50766990 |
| 5 | rs2344182 | 150253104 | 6.38E−13 | IRGM | 5 | 150226085-150228231 |
| 16 | rs2058813 | 50129454 | 1.40E−12 | NOD2 | 16 | 50727507-50766990 |

The clumping function in PLINK and the in-sample LD to clump genome-wide variants into independent association signals was used. For non-MHC regions, the clumping was performed with a radius of 250Kbp and pairwise R2>0.2. For the MHC region, clumping was performed 3 times with a radius of 5 Mb (covering the full MHC region) and pairwise R2>0.1 for the first time and pairwise R2>0.05 for the next two times (For a total of three times). P-value thresholds were set to 0.1, 0.01, and 0.001, leading to 5,502, 1,398, and 495 clumps for non-MHC regions and 88, 65, 54 for MHC regions, respectively.

Polygenic Risk Score

For each individual, the polygenic risk score (PRS) was calculated as/log(OR)*G, in which OR is the odds ratio from the association analysis for the clumped indexed variants and G is the genotype dosage. To calculate the score for a specific locus, a flanking region 300 kbp up- and down-stream was added. The score for NOD2 used fine-mapped putatively causal variants reported in Huang, H. et al. Fine-mapping inflammatory bowel disease loci to single-variant resolution. Nature 547, 173-178 (2017).

Variance Explained

The binomial model was used to calculate the variance explained by factors of interest. In the models, the diagnosis (CD or UC) was regarded as a dependent variable. PRS, serology markers, and/or smoking status were regarded as independent variables. The baseline model was calculated using the first ten principal components and the intercept, and the alternative model with the factors of interest (PRS, serology and/or smoking) added to the baseline model. The nagelkerke pseudo R2 was computed by comparing the alternate model with the baseline model.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of diagnosing a subject with an inflammatory bowel disease, the method comprising:
(a) assaying a biological sample of the subject to generate a genetic dataset comprising genetic data;
(b) processing the genetic dataset at a genetic locus to determine quantitative measure of the genetic locus, wherein the genetic locus comprises a gene associated with Crohn's disease, thereby producing a Crohn's disease profile of the biological sample of the subject; and
(c) applying a prediction model to the Crohn's disease profile to diagnose the inflammatory bowel disease in the subject, or a likelihood that the subject will develop the inflammatory bowel disease.

2. The method of claim 1, wherein the genetic locus is at a gene comprising NOD2, a MHC locus, PTGER4, ATG16L1, IL23R, PHTF1, IRF1, HNF4A or RNF186, or any combination thereof.

3. The method of claim 1, further comprising:
(d) assaying the biological sample of the subject to generate a serological dataset comprising serology data;
(e) processing the serological data set comprising a presence or a level of a serological marker, thereby refining the Crohn's disease profile of the biological sample of the subject; and
(f) applying the prediction model to the Crohn's disease profile refined in (e) to diagnose the inflammatory bowel disease in the subject, or a likelihood that the subject will develop the inflammatory bowel disease.

4. The method claim 3, wherein the serological marker comprises anti-Saccharomyces Cerevisiae antibodies (ASCA IgG and IgA), perinuclear anti-nuclear cytoplasmic antibody (pANCA), anti-flagellin (anti-CBir1), anti-outer membrane porin C (anti-OmpC) or anti-Pseudomonas fluorescens-associated sequence I2 (anti-I2), or a combination thereof.

5. The method of any one of claims 1-4, wherein applying the prediction model is performed to further diagnose the inflammatory bowel disease location comprising an ileum, a colon, or ileocolonic region of an intestine of the subject.

6. The method of any one of claims 1-5, further comprising administering to the subject a Crohn's disease therapy, provided the inflammatory bowel disease diagnosed in the subject is Crohn's disease.
